(19) United States
(12) Patent Application Publication
(10) Pub. No.: US 2011/0040026 A1
(43) Pub. Date: Feb. 17, 2011

(54) Monomers, Polymers, Dispersions and Inks

(76) Inventors: Owen Roger LOZMAN, Manchester (GB); Richard Thomas WILLIAMS, Manchester (GB)

Correspondence Address: MORGAN LEWIS & BOCKIUS LLP, 1111 PENNSYLVANIA AVENUE NW, WASHINGTON, DC, 20004, US

(21) Appl. No.: 12/845,932

(22) Filed: Jul. 29, 2010

(30) Foreign Application Priority Data
Aug. 11, 2009 (GB) 0913925.4

(51) Int. Cl. C08L 33/06; C07C 69/52; C08F 20/10; C09D 11/00; C07C 67/00

(52) U.S. Cl. 524/559; 560/190; 526/318; 560/204

(57) ABSTRACT

A compound of Formula (1) or a salt thereof:

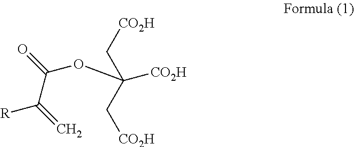

wherein R is H, methyl or ethyl.

### TECHNICAL FIELD

The present invention relates to novel monomers containing a citryl group and to polymers obtainable by polymerising said monomers. The present invention also relates to a process for preparing a dispersion of a pigment using said polymer. The present invention further relates to inks and especially ink jet printing inks.

### INTRODUCTION

Pigment-based inks generally contain a pigment dispersed in a liquid vehicle. In contrast to dye-based inks the pigment is insoluble in the liquid vehicle.

For pigment-based inks it is particularly desirable to obtain high optical density (OD), especially when the ink is printed onto plain paper. It is also desirable to readily obtain an ink containing a fine (sub micron) dispersion of the pigment particles in the liquid vehicle. Such inks are also desirably colloidally stable during storage or use (e.g. printing). That is to say, that desired inks preferably show little or no flocculation or aggregation of the pigment particles during storage or use.

In pigment-based inks the pigment particles are often colloidally stabilised by means of a polymer which acts as a dispersant.

In our own studies on dispersant stabilised pigment-based inks we have found that it is particularly difficult to prepare inks which simultaneously exhibit good colloidal stability and high OD on plain paper. For example, we have found that dispersant stabilised pigment inks known in the art having a high colloidal stability provide a low OD when printed on to plain paper and vice versa.

Commercially, there still remains a need for inks which solve, at least in part, one or more of the abovementioned problems.

### PRIOR ART

A few documents appear, at face value, to have previously prepared citryl (meth)acrylate. Specifically, U.S. Pat. No. 5,660,851, EP 0747343, the Journal of Applied Polymer Science, 94, 2004, pages 57-64 and Polymer International 50, 2001, 119-128. Notably, none of these documents disclosed the use of these monomers for preparing inks.

Importantly however, the NMR spectra provided in these documents were not consistent with the spectra for the intended product. In addition, we repeated the exact synthetic methods disclosed in these documents and several variations thereof but were unable to detect any of the desired citryl (meth)acrylate monomer in the product. Methods such as NMR clearly indicated that, in all cases, no detectable amount of the desired product had been made.

Thus we are firmly of the opinion that these documents do not provide an enabling disclosure of the citryl (meth)acrylate monomer and in fact make something structurally very different.

### Our Studies

The inventors set out to devise a new synthetic method to prepare citryl containing monomers. Clearly, this was difficult as many others had failed in the past. After much work the inventors successfully prepared citryl group containing monomers. The NMR spectra of the products were perfectly consistent with the intended structures. Thus, we consider that this is the first disclosure of a synthetic method which successfully prepares these novel citryl containing monomers.

### FIGURES

FIG. 1—Shows the proton NMR spectra of citryl methacrylate as predicted by the computer software named ACD/HNMR predictor (v9.02).

FIG. 2—Shows a 300 MHz proton NMR spectra of citryl methacrylate in dimethyl sulfoxide (DMSO) as prepared in accordance with the present invention.

FIG. 3—Shows a 300 MHz proton NMR spectra of citryl methacrylate in dimethyl sulfoxide (DMSO) after shaking with trifluoro acetic acid (TFA) again as prepared in accordance with the present invention.

FIG. 4—Shows the proton NMR spectra of citryl methacrylate as disclosed in Polymer International, 50, 2001 at page 123.

FIG. 5—Shows a 300 MHz proton NMR spectra of the product we obtained using the synthetic method described in the Polymer International document to prepare citryl methacrylate.

FIG. 6—Shows the proton NMR spectra of citryl acrylate as disclosed in EP 0747343 at page 32, in FIG. 8.

FIG. 7—Shows a 300 MHz proton NMR spectra of the product we obtained using the synthetic method described in EP 0747343, Example 4 at page 11.

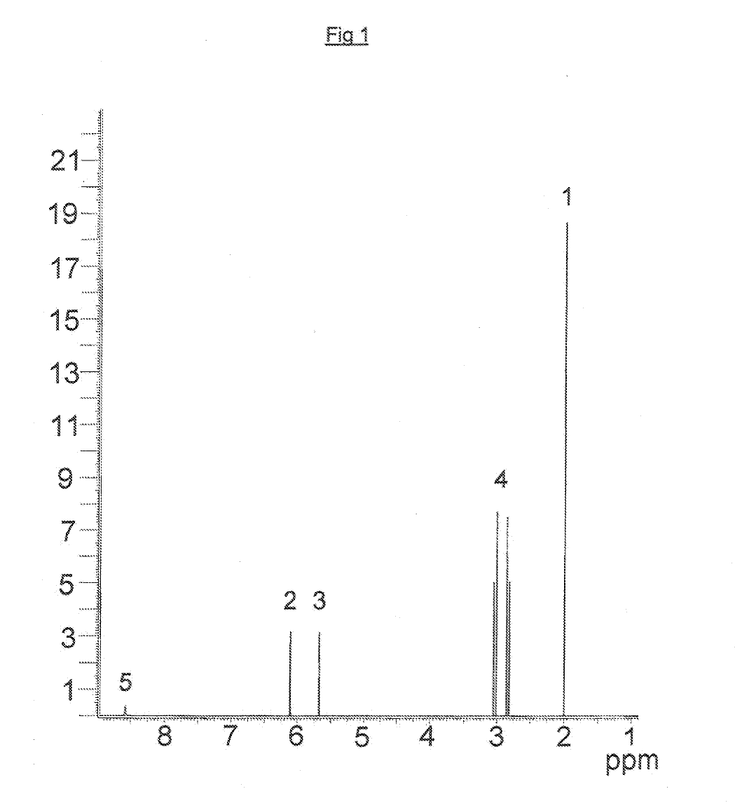

The computer predicted proton NMR spectra of citryl methacrylate is as indicated in FIG. 1.

The different proton environments in citryl methacrylate are as shown in Formula (A):

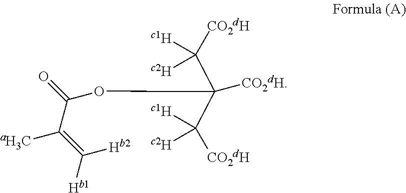

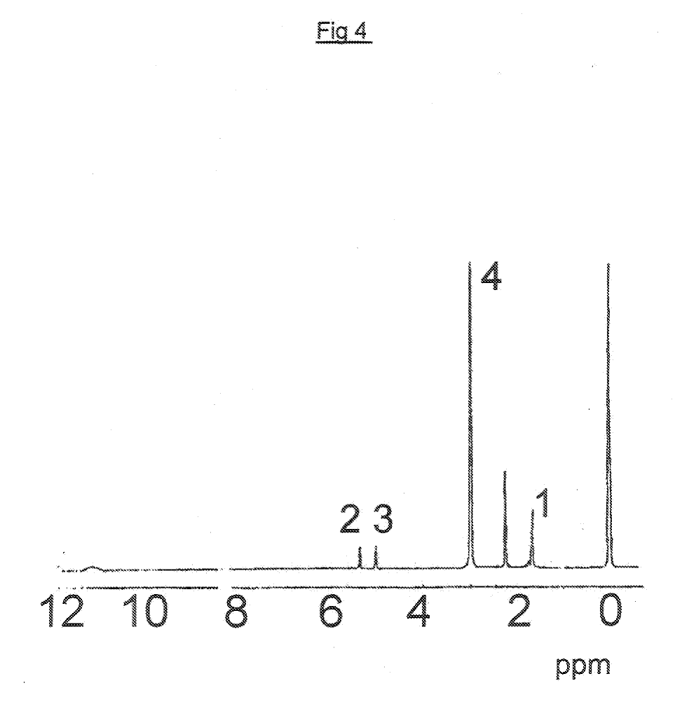

The references in FIGS. 1 and 4 correspond to the protons in the compound of Formula (A) as follow:

1—^{a}H methyl protons;

2—^{b2}H vinyl proton;

3—^{b1}H vinyl proton;

4—c¹H and c²H methylene protons;

5—^{d}H carboxylic acid proton.

Disregarding the carboxylic acid protons (^{d}H) then, broadly speaking three different types of proton environment can be seen. The methyl-^{a}H protons, the vinyl-^{b}H protons and the methylene protons-^{c}H in the citryl group.

The three ^{a}H protons in the methyl group are expected to show as a singlet at a chemical shift value of about 2 ppm.

The two ^{b}H protons in the vinyl group are of two types ^{b1}H and ^{b2}H due to the lack of rotation about the vinyl C═C bond. These two protons would be expected to show as two peaks at a chemical shift value of about 6 ppm.

The four ^{c}H methylene protons in the citryl group are not equivalent and are of two types ^{c1}H and ^{c2}H. These are diastereotopic protons and the non-equivalence originates from the pro chiral centre of the citric acid group. These four ^{c}H protons would be expected to show as four peaks in the form of a pair of doublets and at a chemical shift value of about 3 ppm. Integration of the area under the peaks would be expected to show a ratio of ^{a}H:^{b}H:^{c}H protons of 3:2:4. The actual value taken from our NMR was 2.92:1.96:4.19.

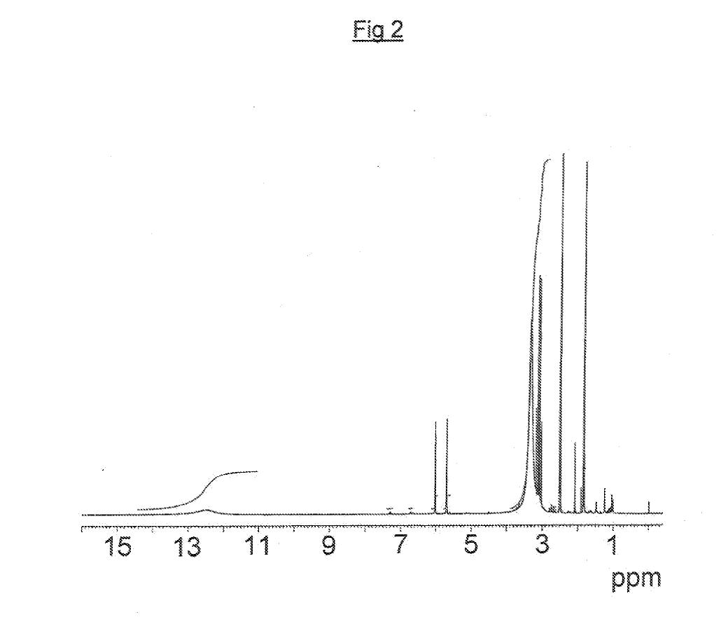

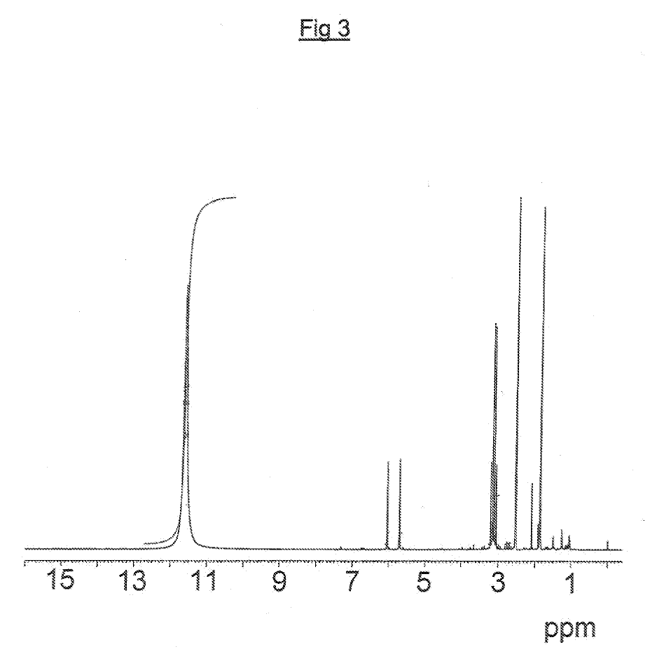

FIGS. 2 and 3 show the experimental proton NMR spectra for citryl methacrylate as prepared in the present invention. The spectra is fully consistent with that predicted for the chemical structure of citryl methacrylate as can be seen by comparison to FIG. 1. Thus, we concluded that citryl methacrylate had been successfully prepared. The trifluoroacetic acid (TFA) shake used in FIG. 3 helps to remove the water peak which partially obscured the ^{c}H diastereotopic protons in FIG. 2. In FIGS. 2 and 3 the carboxylic acid protons predicted are not observed. This is typical for carboxylic acid protons. In FIGS. 2 and 3 the peak at 2.5 ppm is from the solvent DMSO.

FIG. 4 shows the proton NMR spectra for citryl methacrylate as disclosed in the Polymer International document at page 123. It is apparent that the spectra is not consistent with the expected spectra. Specifically, the integration shows that ^{a}H and ^{c}H protons are not present in a ratio of 3:4 respectively. The values from the integration would seem to be approximately 1:4. Importantly, the methylene citryl protons ^{c}H show no splitting pattern as expected from the diastereotopic protons. Clearly, this NMR spectra is not that of the intended product but of something entirely different.

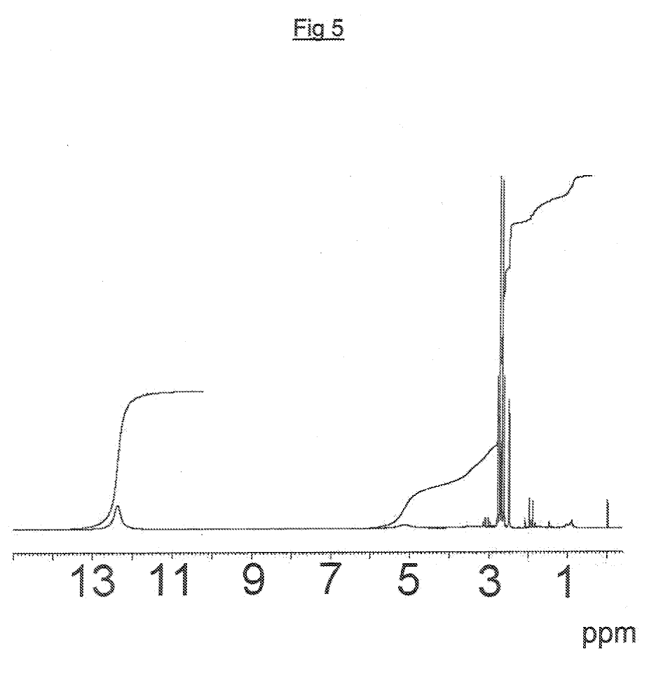

We attempted to prepare citryl methacrylate by the method described in Polymer International, 50, 119-128, 2001. FIG. 5 shows the proton NMR we experimentally obtained for the product after following the work up procedure as described. This spectra is again not consistent with the intended product. In particular, the vinyl protons ^{b}H cannot be seen, the signal at 3 ppm where the ^{c}H proton should be is very weak. Additionally the integration of ^{c}H:^{a}H is not in the ratio of 4:3. Also we found that the integration ratios of ^{c}H and ^{a}H seemed to depend on the extent of work up which strongly suggests that they are groups in different molecules. The main features in FIG. 5 at approximately 2.8 ppm are protons from citric acid.

The methods described in the Journal, of Applied Polymer Science, Vol 94, pages 57 to 64 (2004) are the same as those for the Polymer International publication.

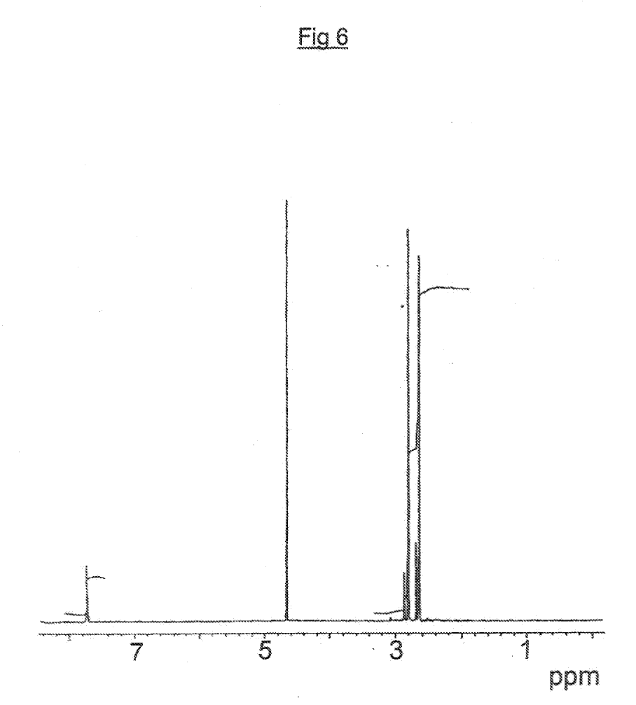

FIG. 6 reproduces the proton NMR spectra as disclosed in EP 0747343 A1 at page 32, FIG. 8 purportedly for citryl acrylate as per Example 4 on page 11. The first thing to be noted is that there are no vinyl ^{b}H protons at approximately 6 ppm. Thus, whatever the product is it carries no vinyl group, so it is not a monomer. Also the splitting pattern of the citryl methylene protons ^{c}H is not correct. Thus, we concluded that EP 0747343 did not successfully prepare citryl acrylate.

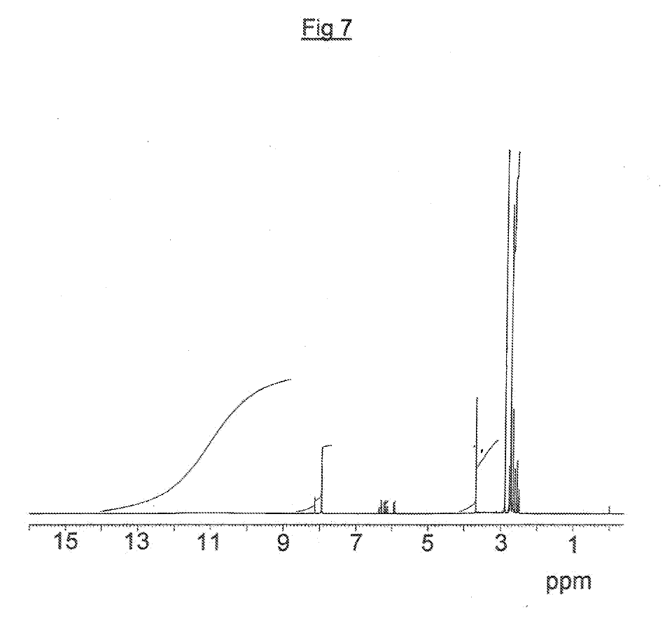

We attempted to repeat the synthetic method as described in EP 0747343 at page 11, Example 4. The 300 MHz proton NMR spectra of the product is as shown in FIG. 7. Again we note that the product contains no vinyl, groups which means, the trans esterification reaction was unsuccessful.

### THE INVENTION

In our own diligent studies we later found that polymers containing citryl groups worked very effectively as pigment dispersants and could be used to readily prepared sub micron dispersions of pigment particles in a liquid medium. Furthermore, we found that inks containing said dispersions had good OD when printed onto plain paper and had good colloidal stability.

According to a first aspect of the present invention there is provided a compound of Formula (1) or a salt thereof:

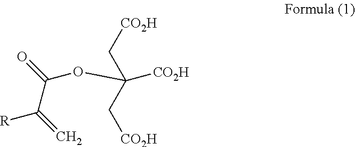

wherein R is H, methyl or ethyl.

### DEFINITIONS

In this description the words “a” and “an” mean one or more unless indicated otherwise. Thus, for example, “a” compound of Formula (1) includes the possibility of there being more than one compound of Formula (1) similarly “a” salt includes the possibility of there being more than one salt.

Citryl methacrylate is a shorthand name for methacryloyl-2-oxy-1,2,3-propanetricarboxylic acid and salts thereof.

Citryl acrylate is a shorthand name for acryloyl-2-oxy-1,2,3-propanetricarboxylic acid and salts thereof.

### R

Preferably, R is H or methyl. These compounds tend to copolymerise most effectively with other ethylenically unsaturated monomers to provide polymers suitable as dispersants.

### Salts

The compound of Formula (1) may be in the form of the free acid or more preferably it may be in the form of a salt. Preferred salt forms include alkali metals, ammonium and substituted ammonium salts including mixtures thereof. Preferred alkali metals include lithium, sodium and especially potassium.

### Synthesis

The prior art documents which purport to synthetically prepare citryl (meth)acrylate monomers do not utilise any protecting groups for the three carboxylic acid groups or salts thereof. In contrast, we have found that protecting these groups is required in order to successfully prepare the desired product.

Accordingly, a preferred process for preparing the compounds of Formula (1) or a salt thereof comprises the steps i) to iii) in the following order:
i) protecting all the carboxylic acid groups or salts thereof in citric acid by means of reacting citric acid or a salt thereof with a protecting agent so as to provide a compound of Formula (2a);

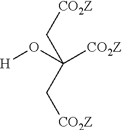

Formula (2a) wherein Z is a protecting group;
ii) esterifying the hydroxy group in the compound of Formula (2a) so as to provide a compound of Formula (2c);

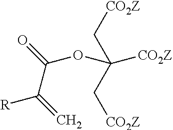

Formula (2c) wherein Z and R are as hereinbefore defined;
iii) deprotecting the CO₂Z ester groups in the compound of Formula (2c) as prepared in step ii).

Preferably, the citric acid is in the form of the free acid prior to the protection reaction in step i).

### The Protection Step i)

The protection step may be carried out using any suitable protecting agent. Examples of protecting agents include organic silanes and especially alcohols. Preferred alcohols include C₁₋₁₀ branched, cyclic and linear aliphatic alcohols and aromatic alcohols. Of these, we have found that benzyl alcohol is a particularly effective protecting agent. Of the organic silanes trialkyl halo silanes are especially preferred.

The protection reaction is preferably performed in a liquid medium. When an alcohol is used as the protecting agent then the preferred liquid media include xylenes, benzene and especially toluene. When alcohols are used as the protecting agent the protection reaction is preferably carried out at a temperature of from 100 to 170° C. and especially from 100 to 150° C. Temperatures around 130° C. are especially suitable. Such temperatures are preferably maintained for a period of from 8 to 30 hours, especially around 24 hours. A catalyst is preferably used for the protection reaction. The catalysts may be a transition metal compound or more preferably an acid. Mineral acids such as sulphuric, phosphoric and especially hydrochloric acid are preferred. Preferably, the protection reaction is conducted in an apparatus fitted with a Dean and Stark trap. In this way the progress of the protection reaction can be monitored by the collection of water.

### Optional Purification

The product of step i) may be used directly in step ii) however it is preferable to purify the protected compound of Formula (2a) prior to step ii).

The liquid medium is preferably removed by evaporation. The crude product remaining after evaporation is preferably purified by dissolution in a water immiscible organic solvent (e.g. ethyl acetate) and washed with water containing a base (e.g. potassium carbonate). The water washing step is preferably repeated several times until any unreacted materials are fully removed. The washed product is preferably dried for example by using molecular beads or an anhydrous metal salt (e.g. magnesium sulphate).

When the protecting agent is volatile, any remaining protecting agent is preferably removed by evaporation or distillation.

Any remaining particulate impurities are preferably removed by centrifugation or filtration.

### The Esterification Step ii)

Preferably, the esterifying compound is of the Formulae (2b1) or (2b2):

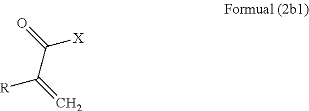

wherein X is a halo and R is as hereinbefore defined;

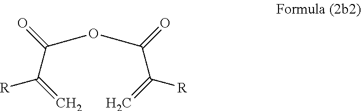

wherein R is as hereinbefore defined.

Preferably, X is chloro.

Preferably, the esterification reaction is performed under anhydrous conditions.

Preferably, the esterifying compound is added slowly to the compound of Formula (2a). Preferably, the esterification reaction is performed at a temperature of from 30 to −30° C., especially from 10 to −10° C. This temperature is preferably maintained for a period of from 30 minutes to 5 hours, especially from 1 to 3 hours.

The reaction is preferably performed in an aprotic organic liquid medium. Preferred liquid media for the estherification reaction include acetonitrile, tetrahydrofuran, diethyl ether, dioxane, dimethylformamide, benzene, toluene, hexane, xylene and carbon tetrachloride and especially dichloromethane. A catalyst is preferably used for the esterification reaction. Preferred catalysts include transition metal compounds and especially organic amine bases. Preferred organic amine bases include trialkylamines and/or N,N-dialkylaminopyridines. Of the trialkylamines triethylamine is preferred. Of the N, N-dialkylaminopyridines 4-dimethylaminopyridine (DMAP) is preferred. We have found that the combination of both triethylamine and DMAP is especially effective as a catalyst mixture.

The product obtained from step ii) can be used directly in step iii) however it is preferable to purify the product of step ii) prior to step iii).

The purification methods are the same as those previously described above in reference to the crude product obtained in step i).

The Deprotection Step iii)

The compound of Formula (2c) may be deprotected by adding a catalyst. Suitable catalysts include acids, bases and transition metal compounds. We have found that palladium compounds are particularly effective for use in the deprotection step iii). A preferred example of which is palladium acetate. The liquid medium for the deprotection step is preferably an aprotic organic liquid medium as hereinbefore defined in step ii). Organic amine bases and especially triethylamine is also helpful in speeding the deprotection reaction. We have also found that the use of an organic silane (preferably trialkyl silane, especially triethyl silane) is especially effective at assisting the deprotection reaction.

In a preferred embodiment the deprotection reaction is performed using a palladium compound, an organic amine base and an organic silane. It is especially preferred to use a mixture of palladium acetate, triethylamine and triethyl silane to best assist the deprotection reaction.

The product of step iii) is a crude product containing the compound of Formula (1) or a salt thereof.

Preferably, the crude product is purified which may be by the same methods as described after step i).

It is possible to use other techniques for final purification such as column chromatography, cross-flow ultrafiltration and recrystallisation.

### Salts

Preferably, the salt of the compounds of Formula (1) is prepared by neutralising the free acid form with a base such as for example lithium, sodium or potassium hydroxide. Amines and substituted amines can also be used as bases. Ion-exchange resins can also be used for preparing the salt forms.

### Preferred Synthesis

A preferred synthetic process comprises the steps i) to iii) in the following order:
i) protecting all the carboxylic acid groups or salts thereof in citric acid by means of reacting citric acid or a salt thereof with benzyl alcohol so as to provide a compound of Formula (2a);

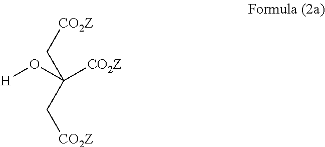

wherein Z is a benzyl group;
ii) esterifying the hydroxy group in the compound of Formula (2a) with a (meth)acryloyl halide (especially chloride) so as to provide a compound of Formula (2c);

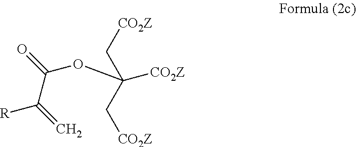

wherein Z and R are as hereinbefore defined;
iii) deprotecting the CO₂Z ester groups in the compound of Formula (2c) as prepared in step ii) in the presence of a palladium compound an organic amine and an organic silane.

### Polymerisation

According to a second aspect of the present invention there is provided a polymer obtained or obtainable by polymerising at least a compound of Formula (1) or a salt thereof according to the first aspect of the present invention. Such polymers comprise repeat units from the compounds of Formula (1) or salts thereof.

The compound of Formula (1) or a salt thereof may be homopolymerised, however the compound of Formula (1) or a salt thereof is preferably copolymerised with other monomers.

Any suitable polymerisation method can be used although we have found that free radical polymerisation is particularly suitable.

Free radical polymerisation is preferably performed using a free radical initiator, which is preferably a redox or thermal initiator. Examples of suitable thermal free radical initiators include peroxides, azos and persulfates.

The polymerisation can be by suspension, bulk, emulsion or more preferably by solution polymerisation. Preferred solvents for solution polymerisation include water, alcohols, ketones, glycols and pyrrolidones. Of these glycols, alcohols and mixtures thereof are especially preferred.

### Copolymerisation

Preferred polymers are obtained by copolymerising a monomer composition comprising:

i) one or more compounds of Formula (1) or salts thereof;
ii) one or more hydrophobic ethylenically unsaturated monomers; and optionally iii) one or more hydrophilic ethylenically unsaturated monomers other than component i).

Preferred polymers comprise from 1 to 50, more preferably from 3 to 25 percent by weight of repeat units from the compound of Formula (1) or a salt thereof.

### Hydrophobic Ethylenically Unsaturated Monomers

Hydrophobic ethylenically unsaturated monomers preferably comprise one or more hydrophobic groups.

Preferred hydrophobic groups are hydrocarbons, fluorocarbons and alkyl siloxanes comprising less than three and more preferably no hydrophilic groups.

Preferably, the hydrophobic ethylenically unsaturated monomers, all have a calculated Log P value of at least 1, more preferably from 1 to 6, especially from 2 to 6.

A review by Mannhold, R. and Dross, K. (Quant. Struct-Act. Relat. 15, 403-409, 1996) describes 14 methods for calculating Log P values of compounds and especially drugs. From this review we prefer the “fragmental methods” and especially the fragmental method implemented by ACD labs software. The calculated Log P of a monomer may be calculated using commercially available computer software, for example using the Log P DB software version 7.04 or a later version of such software (which is available from Advanced Chemistry Development Inc (ACD labs)). Any ionic or ionisable groups are calculated in their neutral (unionised) form. A higher log P value corresponds to a more hydrophobic monomer. We have found the inclusion of such monomers helps provide good dispersant properties.

Preferred hydrophobic ethylenically unsaturated monomers are styrenic monomers (e.g. styrene, alpha methyl styrene), aryl (meth)acrylates (especially benzyl acrylate and benzyl methacrylate), C₁₋₃₀-alkyl (meth)acrylates (especially 2-ethyl hexyl (meth)acrylate and butyl (meth)acrylate), butadiene, (meth)acrylates containing poly(C₃₋₄)alkylene oxide groups, (meth)acrylates containing alkylsiloxane or fluorinated alkyl groups and vinyl naphthalene.

Preferably, at least one of the one or more hydrophobic ethylenically unsaturated monomers is selected from aryl (meth)acrylates and C₁₋₃₀ alkyl (meth)acrylates.

More preferably, all of the one or more hydrophobic ethylenically unsaturated monomers are each independently selected from aryl (meth) acrylates and C₁₋₃₀ alkyl (meth) acrylates.

Especially, at least one of the hydrophobic ethylenically unsaturated monomers is selected from 2-ethyl hexyl (meth)acrylate, lauryl (meth)acrylate, benzyl (meth)acrylate, butyl (meth)acrylate, styrene and methyl (meth)acrylate.

Of all the hydrophobic monomers benzyl (meth)acrylate and 2-ethyl hexyl (meth)acrylate are preferred as these have been found to copolymerise effectively and provide polymers which have good pigment dispersing properties.

Most preferably, the hydrophobic monomer(s) in component ii) is or comprises benzyl methacrylate, 2-ethyl hexyl methacrylate and/or n-butyl methacrylate.

Preferred, polymers comprise from 50 to 99, more preferably from 60 to 97 percent by weight of repeat units from hydrophobic ethylenically unsaturated monomers.

### Hydrophilic Ethylenically Unsaturated Monomers

When present in the monomer composition the hydrophilic ethylenically unsaturated monomers must be other than the compound of Formula (1) or a salt thereof.

The hydrophilic ethylenically unsaturated monomers preferably comprise one or more hydrophilic groups. The hydrophilic group may be non-ionic or ionic, however it is preferred not to incorporate monomers in component iii) containing hydrophilic non-ionic groups as we have found that these groups tend to reduce the final optical density of prints obtained from inks containing such polymeric dispersants. Hydrophilic non-ionic groups which are preferably absent include polyethyleneoxy and hydroxy groups. Thus, preferred polymers do not have any repeat units from polyethyleneoxy functional (meth)acrylates or hydroxy functional (meth)acrylates.

Ionic hydrophilic groups may be cationic but are preferably anionic.

The anionic groups are preferably acidic groups of which preferred examples include phosphonic acid, phosphoric/polyphosphoric acid esters, sulfonic acid, sulphuric acid esters and especially carboxylic acid groups including salts thereof.

Preferably, at least one of the hydrophilic ethylenically unsaturated monomers containing carboxylic acid groups or salts thereof is selected from acrylic acid, methacrylic acid, beta carboxy ethyl acrylate, itaconic acid, crotonic acid, maleic acid, maleic anhydride (followed by ring opening) or fumaric acid.

Preferably the monomers in component iii) all have a calculated Log P value of less than 1, more preferably from 0.99 to −2, especially from 0.99 to 0 and most especially from 0.99 to 0.5 when calculated in the un-neutralised (e.g. free acid) form.

Preferred, polymers comprise from 0 to 25, more preferably from 0 to 15 percent by weight of repeat units from hydrophilic ethylenically unsaturated monomers.

Preferably, the polymer comprises no repeat units from monomers, in component iii) as described above.

### Preferred Polymer Composition

Preferred polymers are obtained by copolymerising a monomer composition comprising:
i) 1 to 50 parts, more preferably 3 to 25 parts of one or more compounds of Formula (1) or salts thereof;
ii) 50 to 99 parts, more preferably 60 to 97 parts of one or more hydrophobic ethylenically unsaturated monomers; and
iii) 0 to 25 parts, more preferably 0 to 15 parts of one or more hydrophilic ethylenically unsaturated monomers other than component i).
   wherein all the parts are by weight and the sum of the parts i)+ii)+iii) adds up to 100.

In some cases, component iii) is present in the monomer mixture in less than 5 parts and especially component iii) is absent from the monomer mixture.

### Polymer Characteristics

Preferably, the polymers described above have an acid value of from 30 to 300 mg KOH/g, more preferably from 50 to 150 mg KOH/g and especially from 60 to 120 mg KOH/g.

Preferably, the polymer has a weight averaged molecular weight of from 1,000 to 1,000,000, more preferably from 5,000 to 200,000 and especially from 20,000 to 150,000. The weight averaged molecular weight is preferably measured by gel permeation chromatography (GPC).

The polymer may have acid groups from the compound of Formula (1) in the form of the free acid or more preferably in the form of a salt. The preferred salt forms are as hereinbefore described for the compound of Formula (1) itself.

Preferably, the polymer has no hydrophilic groups other than carboxylic acid groups or salts thereof.

### Dispersions

The polymers according to the second aspect of the present invention are particularly effective at dispersing pigments in liquid media.

According to a third, aspect of the present invention there is provided a dispersion comprising a polymer according to the second aspect of the present invention; a pigment and a liquid medium.

### Pigment

The pigment may comprise and preferably is an inorganic or organic pigment material or mixture thereof which is insoluble in the liquid medium.

Preferred organic pigments include, for example, any of the classes of pigments described in the Colour Index International, Third Edition, (1971) and subsequent revisions of, and supplements thereto, under the chapters headed “Pigments”. Examples of organic pigments include those from the azo (including disazo and condensed azo), thioindigo, indanthrone, isoindanthrone, anthanthrone, anthraquinone, isodibenzanthrone, triphendioxazine, quinacridone and phthalocyanine series, especially copper phthalocyanine and its nuclear halogenated derivatives, and also lakes of acid, basic and mordant dyes. Preferred organic pigments are phthalocyanines, especially copper phthalocyanine pigments, azo pigments, indanthrone, anthanthrone and quinacridone pigments. Preferred organic pigments include C.I. Pigment Red 122, C.I. Pigment Blue 15:3 and C.I. Pigment Yellow 74.

Preferred inorganic pigments include carbon black, titanium dioxide, aluminium oxide, iron oxide and silicon dioxide.

In the case of carbon black pigments, these may be prepared in such a fashion that some of the carbon black surface has oxidized groups (e.g. carboxylic acid and/or hydroxy groups). However, the amount of such groups is preferably not so high that the carbon black may be dispersed in water without the aid of a dispersant.

Preferably, the pigment is a cyan, magenta, yellow or black pigment.

The pigment may be a single chemical species or a mixture comprising two or more chemical species (e.g. a mixture comprising two or more different pigments). In other words, two or more different pigments may be used in the process of the present invention. Where two or more pigments are used these need not be of the same colour or shade.

Preferably, the pigment is not dispersible in an aqueous liquid medium without the aid of a dispersant, i.e. the presence of a dispersant is required to facilitate dispersion. Preferably, the pigment is not chemically surface treated, for example by having ionic groups covalently bonded to its surface (especially not —CO₂H or —SO₃H).

Preferably, the pigment is carbon black or an organic pigment.

### Liquid Media

Preferably, the liquid medium is or comprises water.

Preferably the liquid media contains water and organic solvent in the weight ratio of 99:1 to 1:99, more preferably 99:1 to 50:50 and especially 95:5 to 70:30.

Preferred organic solvents are water-miscible organic solvents and mixtures of such solvents. Preferred water-miscible organic solvents include C₁₋₆-alkanols, preferably methanol, ethanol, n-propanol, isopropanol, n-butanol, sec-butanol, tert-butanol, n-pentanol, cyclopentanol and cyclohexanol; linear amides, preferably dimethylformamide or dimethylacetamide; ketones and ketone-alcohols, preferably acetone, methyl ether ketone, cyclohexanone and diacetone alcohol; water-miscible ethers, preferably tetrahydrofuran and dioxane; diols, preferably diols having from 2 to 12 carbon atoms, for example pentane-1,5-diol, ethylene glycol, propylene glycol, butylene glycol, pentylene glycol, hexylene glycol and thiodiglycol and oligo- and poly-alkyleneglycols, preferably diethylene glycol, Methylene glycol, polyethylene glycol and polypropylene glycol; triols, preferably glycerol and 1,2,6-hexanetriol; mono-C₁₋₄-alkyl ethers of diols, preferably mono-C₁₋₄-alkyl ethers of diols having 2 to 12 carbon atoms, especially 2-methoxyethanol, 2-(2-methoxyethoxy)ethanol, 2-(2-ethoxyethoxy)-ethanol, 2-[2-(2-methoxyethoxy)ethoxy]ethanol, 2[2-(2-ethoxyethoxy)-ethoxy]-ethanol and ethyleneglycol monoalkylether; cyclic amides, preferably 2-pyrrolidone, N-methyl-2-pyrrolidone, N-ethyl-2-pyrrolidone, caprolactam and 1,3-dimethylimidazolidone; cyclic esters, preferably caprolactone; sulphoxides, preferably dimethyl sulphoxide and sulpholane. Preferably, the liquid medium comprises water and 2 or more, especially from 2 to 8, water-miscible organic solvents.

Especially preferred water-miscible organic solvents for the ink are cyclic amides, especially 2-pyrrolidone, N-methyl-pyrrolidone and N-ethyl-pyrrolidone; diols, especially 1,5-pentane diol, ethyleneglycol, thiodiglycol, diethyleneglycol and triethyleneglycol; and mono-C₁₋₄-alkyl and di-C₁₋₄-alkyl ethers of diols, more preferably mono-C₁₋₄-alkyl ethers of diols having 2 to 12 carbon atoms, especially 2-methoxy-2-ethoxy-2-ethoxyethanol.

Examples of further suitable liquid media comprising a mixture of water and one or more organic solvents are described in U.S. Pat. No. 4,963,189, U.S. Pat. No. 4,703,113, U.S. Pat. No. 4,626,284 and EP 4,251,50A.

### Process for Preparing a Dispersion

According to a fourth aspect of the present invention there is provided a process for preparing a dispersion of a pigment in a liquid medium comprising the steps:
i) providing a mixture comprising the pigment, the liquid medium and a polymer according to the second aspect of the present invention;
ii) mechanically treating the mixture in i) so as to disperse the pigment in the liquid medium.

The pigment and the liquid medium are as hereinbefore described.

Preferably, the volume averaged particle size of the pigment particles after the mechanical treatment is from 30 nm to 500 nm, more preferably from 30 nm to 300 nm and especially from 50 nm to 150 nm.

Preferably, the mechanical treatment is or comprises bead milling, bead shaking, ultrasonication, microfluidization, high pressure homogenisation or a combinations thereof. Of these we find bead milling is particularly effective.

Preferably, the pigment is relatively concentrated during the dispersion process. Preferably, during the dispersion process the pigment is present in compositions comprising pigment, liquid medium and polymer at from 10 to 60%, more preferably 10 to 20% by weight.

### Encapsulated/Cross-Linked Dispersant

Preferably, the polymer according to the second aspect of the present invention is cross-linked in the presence of the pigment and the liquid medium so as to encapsulate the pigment. This has the effect of encapsulating pigment particles which are dispersed in the liquid medium.

This can be achieved by adding a cross-linking agent to a dispersion according to the fourth aspect of the present invention. Preferably, the dispersion and cross-linking agent are heated to a temperature of from 30 to 120° C., more preferably from 40 to 100° C. to speed the cross-linking reaction.

The cross-linking site in the polymer may be any hydrophilic group however it is preferred to cross-link via carboxylic acid groups or salts thereof.

Suitable cross-linking agents which effectively cross-link carboxylic acid functional polymers include titanates, isocyanates, aziridines, carbodiimides, oxazolines, n-methylol containing compounds and especially epoxides.

Examples of epoxy cross-linking agents are disclosed in PCT patent publication WO2006/064193 at page 11, lines 1 to 15.

### Purification of the Dispersion

Preferably; the dispersion according to the third aspect or as prepared by the process according to the fourth aspect of the present invention is purified. The purification can be by any suitable method including microfiltration, deionizer resins, centrifugation followed by decantation and washing. A preferred method is membrane filtration especially ultrafiltration. Preferred ultrafiltration membranes have a pore size of about 0.1 microns. Preferably, the dispersion is washed with from 5 to 50 volumes of purified water based on the volume of the dispersion. Preferably, the water used in the ultrafiltration process is deionized, distilled or has been purified by reverse osmosis. A preferred method of assessing when the dispersion has been sufficiently purified is to measure the conductivity of the permeate stream from the ultrafiltration stage and to continue adding further volumes of pure water until the permeate stream has a conductivity of less than 100 μS/cm, more preferably less than 50 μS/cm. The ultrafiltration is preferably performed on a dispersion which has from 10 to 15% by weight of pigment in the dispersion. We have found that purification can in some instances provide final dispersions and inks having further improved OD when printed onto plain paper.

### Inks and Ink Additives

According to a fifth aspect of the present invention there is provided an ink comprising a dispersion according to the third aspect of the present invention or as prepared by the fourth aspect of the present invention and one or more additives.

Preferably, the ink is or comprises water.

Preferred additives for preparing inks include those selected from viscosity modifiers, pH buffers, metal chelating agents, surfactants, corrosion inhibitors, biocides, dyes, water miscible organic solvent(s) and/or kogation reducing additives.

### Inks and Ink Jet Printing Inks

The ink according to the fifth aspect of the present invention is preferably an ink jet printing ink.

Preferably, the ink has a viscosity of less than 50 mPa·s, more preferably less than 30 mPa·s and especially less than 15 mPa·s, when measured at a temperature of 25° C.

Preferably, the ink has a surface tension of 20 to 65 dynes/cm, more preferably 30 to 60 dynes/cm, when measured at a temperature of 25° C.

The pH of the ink is preferably from 4 to 11, more preferably from 7 to 10.

When the ink is to be used as ink jet printing ink, the ink preferably has a concentration of halide ions of less than 500 parts per million, more preferably less than 100 parts per million. It is especially preferred that the ink has less than 100, more preferably less than 50 parts per million of divalent and trivalent metals. Parts per million as used above refers to parts by weight relative to the total weight of the ink. These low concentrations of ions in the resultant ink can be achieved by the abovementioned purification step.

Preferably the process for making the ink includes a step for removing particles having a particle size of more than 1 micron in diameter, for example by filtration or centrifugation. Preferably the ink has less than 10%, more preferably less than 2% and especially less than 1% by weight of particles having a size of greater than 1 micron in diameter.

Preferably, the amount of pigment in the ink is from 0.1 to 15%, more preferably from 1 to 10% and especially from 3 to 10% by weight.

### Applications

The process according to the fourth aspect of the present invention is especially suitable for preparing aqueous pigment dispersions for use in an ink jet printing ink. In addition the aqueous pigment dispersions may be used in gravure or lithographic inks, paints, tints, cosmetics, thermoplastics and thermosets.

### Use

According to a sixth aspect of the present invention there is provided the use of the polymer according to the second aspect of the present invention for preparing an ink jet printing ink comprising a pigment, a liquid media and the polymer.

Preferably, this use is for the technical purpose of providing an ink jet printing ink which provides higher optical density when printed onto plain paper.

### Fixing Agents

The ink jet printing inks may in some embodiments be used with papers which comprise fixing agents to improve, for example, wet fastness, optical density or to reduce colour bleeding. In another embodiment ink jet printing inks containing aqueous pigment dispersions prepared by the process of the present invention may be used alongside fixing agents. For example, an ink jet printer cartridge might comprise an ink as described above in one chamber and a liquid comprising a fixating agent in a further chamber. In this way the ink jet printer may apply the ink and the fixing agent to a substrate.

Fixing agents are known in the art and include such things as metal salts, acids and cationic materials.

The invention is further illustrated by the following Examples in which all parts and percentages are by weight unless otherwise stated.

### EXAMPLES

The Experiments described below may be scaled as is required the amounts have been expressed in gms and in some cases mmols.

### 1. Preparation of Citric Acid Methacrylate (CAMA)

### Stage 1.1 Preparation of Tribenzyl Citrate (Protection Step)

Citric acid monohydrate (75 g, 357 mmols), benzyl alcohol (155 g, 1429 mmols), toluene (200 ml) and concentrated hydrochloric acid (4 drops as catalyst) were stirred together in a flask to form a reaction mixture.

The reaction mixture was heated to and maintained at a temperature of 130° C. whilst the water generated was collected in a Dean and Stark trap. Heating was continued for 24 hours by which time about 23 ml of water had been collected. Toluene was removed from the mixture by rotary evaporation. The resulting oil was dissolved in ethyl acetate (400 ml) to form an organic phase which was washed three times with a saturated aqueous potassium carbonate solution (400 ml). The organic phase was dried over anhydrous magnesium sulphate, filtered and concentrated by evaporation. Any remaining benzyl alcohol was removed by vacuum distillation at 125° C. and 0.4 mbar.

The organic phase was further purified by mixing it with acetone followed by filtration to remove any insoluble impurities. The organic phase was then concentrated by evaporating off the acetone.

The expected compound (tribenzyl citrate) was isolated as a yellow oil 101 g (61%).

The proton and carbon NMR spectra were consistent with the expected compound.

¹H NMR (300 MHz DMSO) δ 7.31 (m, 15H, aromatic), 5.04 (s, 4H, outer benzyl CH₂), 5.01 (s, 2H, central benzyl CH₂), 3.01 (d, 2H, J=15.3 Hz, citrate) and 2.87 ppm (d, 2H, J=15.3 Hz, citrate). ¹³C NMR (75 MHz DMSO) δ 172.31, 169.01, 135.79; 135.59, 128.29, 128.26, 127.93, 127.87, 124.76, 73.09, 66.33, 65.64 and 42.99 ppm (13 signals observed, 14 expected).

### Stage 1.2 Esterification

Tribenzyl citrate as prepared in stage 1.1 (4.00 g, 8.65 mmols), triethylamine (0.97 g, 9.51 mmols), 4-dimethylaminopyridine (DMAP) (0.21 g, 1.73 mmols) and anhydrous dichloromethane DCM (20 ml) were mixed together in a flask under a nitrogen gas inert atmosphere.

The mixture was cooled to 0° C. using an ice-salt bath. Methacryloyl chloride (0.99 g, 9.51 mmols) was added drop-wise ensuring the temperature was kept below 2° C. On completion of addition, the resulting yellow/brown cloudy product mixture was stirred at 0° C. for 2 hours, then at 25° C. overnight.

Aqueous potassium carbonate solution (50 ml) was slowly added to the product mixture and stirred for 30 minutes at room temperature before extracting the product with three volumes of DCM (100 ml). The combined DCM organic phases were dried over anhydrous magnesium sulphate, filtered and concentrated by evaporation.

The DCM organic phase was purified by column chromatography using 50% DCM in hexane as eluent.

The tribenzyl citrate methacrylate was isolated as a colourless oil 2.95 g (64%).

The proton and carbon NMR spectra were consistent with the expected compound.

¹H NMR (300 MHz DMSO) δ 7.31 (m, 15H, aromatic), 5.95 (bs, 1H, vinyl), 5.63 (t, 1H, J=1.5 Hz, vinyl), 5.09 (s, 2H, central benzyl CH₂), 5.04 (s, 4H, outer benzyl CH₂), 3.37 (d, 2H, J=15.2 Hz, citrate), 3.29 (d, 2H, J=15.2 Hz, citrate) and 1.72 ppm (s, 3H, methacrylate CH₃). ¹³C NMR (75 MHz DMSO) δ 168.39, 167.94, 165.07, 135.54, 135.06, 134.89, 128.31, 128.12, 128.04, 127.99, 127.80, 127.29, 77.95, 67.05, 66.05, 38.86 and 17.42 ppm (17 signals observed, 18 expected).

### Stage 1.3 Deprotection

Palladium acetate (0.95 g, 4.2 mmols), anhydrous DCM (100 ml) and triethylamine (1.5 g, 14.2 mmols) were stirred together in a flask under a nitrogen atmosphere.

Triethylsilane (14.8 g, 1272 mmols) was added drop-wise to the flask ensuring the reaction temperature was kept below 24° C. The resulting dark brown/black solution was stirred at about 25° C. for 15 minutes.

Tribenzyl citrate methacrylate as prepared in stage 1.2 (15.0 g, 28.3 mmols) dissolved in DCM (25 ml) was added drop-wise to the flask contents, again ensuring the temperature was kept below 24° C., this formed a reaction mixture. The reaction mixture was stirred overnight at room temperature.

The resulting product was diluted with aqueous potassium carbonate solution (200 ml), then hydrophobic impurities were extracted with three portions of DCM (250 ml) and discarded. The aqueous phase was acidified to pH 3 using 3 M HCl before the product was extracted with three portions of diethyl ether (400 ml). The combined diethyl ether organic phases were dried over anhydrous magnesium sulphate, filtered and concentrated by evaporation.

Citric acid methacrylate was isolated as a colourless oil which crystallised slowly.

The resulting off white crystalline solid was then stirred in DCM and collected by filtration 5.7 g (77%).

The proton and carbon NMR spectra, were consistent with the intended product.

¹H NMR (300 MHz DMSO) δ 12.50 (bs, 3H, CO₂H), 6.04 (s, 1H, vinyl), 5.72 (t, 1H, J=1.6 Hz, vinyl), 3.16 (d, 2H, J=15.2 Hz, citrate), 3.06 (d, 2H, J=15.2 Hz, citrate) and 1.84 ppm (s, 3H, methacrylate CH₃). ¹³C NMR (75 MHz DMSO) δ 170.24, 170.04, 165.04, 135.56, 126.61, 78.09, 38.47 and 17.68 ppm.

### 2. Copolymerisation of Citric Acid Methacrylate

Citric acid methacrylate as prepared in stages 1.1 to 1.3 above (20 molar equivalents), benzyl methacrylate (80 molar equivalents) and a chain transfer agent-butyl 3-mercaptopropionate (0.8 molar equivalents based on total monomer) were dissolved in isopropyl alcohol (IPA) to give a 40% solution (monomer weight to total solution weight). The solution was poured into a reaction vessel and stirred under a nitrogen atmosphere.

An initiator Trigonox™ 21S (1 weight percent based on total monomer plus chain transfer agent) was added into the reaction vessel, and the contents were heated to a temperature of 85° C. under a nitrogen atmosphere and with continuous stirring. The temperature of 85° C. was maintained for 4 hours. A second initiator charge of Trigonox™ 21S (1 weight percent based on total monomer plus chain transfer agent) was added and the polymerisation was continued for a further 4 hours still at 85° C.

The reactor contents were then allowed to cool to about 25° C.

IPA was next removed by rotary evaporation to yield a copolymer which was purified by repeated precipitation from a solution of acetone into methanol. Proton NMR showed that the resulting copolymer contained benzyl methacrylate (BzMA) and citric acid methacrylate (LAMA), repeat units. The incorporation of citric acid methacrylate was not 100% effective and the final copolymer had a composition of CAMA/BzMA of 11/89 rather than 20/80.

### 3. Polymeric Dispersant Synthesis

### 3.1 Preparation of Polymeric Dispersant (1)

The ethylenically unsaturated monomers benzyl methacrylate (3.79 g, 21.52 mmoles) and citric acid methacrylate (1.40 g, 5.38 mmoles) were mixed together along with a chain transfer agent butyl 3-mercaptopropionate (0.036 g, 0.22 mmoles). The mixture of ethylenically unsaturated monomers and the chain transfer agent were then dissolved in isopropyl alcohol (7.92 g) to give a 40% by weight solution which was charged into a reactor.

A thermal initiator, Trigonox™ 21S (0.052 g) was then added to the reactor contents, and the contents were stirred continuously whist the temperature was maintained at 80° C. for 4 hours. The reaction was performed using a nitrogen gas atmosphere throughout.

A second charge of thermal, initiator, Trigonox™ 21S (0.052 g), was then added and the polymerisation was continued at a temperature of 80° C. for a further 4 hours, still using a nitrogen gas atmosphere. These steps polymerised the ethylenically unsaturated monomers to prepare the Polymeric Dispersant (D1).

The reactor contents were then cooled to a temperature of 25° C., poured into a rotary evaporator flask and evaporated to dry the Polymeric Dispersant (D1) to about 100% solids. The molecular weights of the Polymeric Dispersant (D1) as measured by gel permeation chromatography using a DMF solvent and polystyrene standards were Mn 45,100 and Mw 102,500. The composition of the polymer prepared was confirmed using proton NMR spectroscopy. Thus, step 3.1 resulted in the preparation of Polymeric Dispersant (D1).

### 3.2 Polymer Neutralisation

The Polymeric Dispersant (D1) prepared in 3.1 above (5.28 g) was diluted to 40% by weight with dipropylene glycol (7.92 g) and neutralised by the addition of a solution containing potassium hydroxide (0.73 g) and water (24.58 g) to give a solids content of 15% by weight. This prepared Neutralised dispersant solution (ND1).

### 3.4 Preparation of Polymeric Dispersant (D2)

The ethylenically unsaturated monomers benzyl methacrylate (4.98 g, 28.25 mmoles) and citric acid methacrylate (1.40 g, 5.38 mmoles) were mixed together along with and a chain transfer agent butyl 3-mercaptopropionate (0.044 g, 0.27 mmoles). The mixture of ethylenically unsaturated monomers and the chain transfer agent were then dissolved in isopropyl alcohol (9.73 g) to give a 40% by weight solution which was charged into a reactor.

A thermal initiator, Trigonox™ 21S (0.064 g) was then added to the reactor contents, and the contents were stirred continuously whist the temperature was maintained at 80° C. for 4 hours. The reaction was performed using a nitrogen gas atmosphere throughout.

A second charge of thermal initiator, Trigonox™ 21S (0.064 g), was then added and the polymerisation was continued at a temperature of 80° C. for a further 4 hours, still using a nitrogen gas atmosphere. These steps polymerised the ethylenically unsaturated monomers to prepare the Polymeric Dispersant (D2).

The reactor contents were then cooled to a temperature of 25° C., poured into a rotary evaporator flask and evaporated to dry thePolymeric Dispersant (D2) to about 100% solids. The molecular weights of the Polymeric Dispersant (D2) as measured by gel permeation chromatography using a DMF solvent and polystyrene standards were Mn 41,000 and Mw 99,200. The composition of the polymer prepared was confirmed using proton NMR spectroscopy. Thus, step 3.4 resulted in the preparation of Polymeric Dispersant (D2).

### 3.5 Polymer Neutralisation

The Polymeric Dispersant (D2) prepared in 3.4 above (6.48 g) was diluted to 40% by weight with dipropylene glycol (9.73 g) and neutralised by the addition of a solution containing potassium hydroxide (0.73 g) and water (29.59 g) to give a solids content of 15% by weight. This prepared Neutralised dispersant solution (ND2).

### 3.6 Preparation of Comparative Polymeric Dispersant Solution (CD1)

The ethylenically unsaturated monomers benzyl methacrylate (18.2 g, 103.3 mmoles) and methacrylic acid (5.0 g, 58.1 mmoles) were mixed together along with and a chain transfer agent butyl 3-mercaptopropionate (0.21 g, 1.3 mmoles). The mixture of ethylenically unsaturated monomers and the chain transfer agent were then dissolved in a liquid mixture of isopropyl alcohol (59.1 g) and dipropylene glycol (35.5 g) to give a 20% by weight solution which was charged into a reactor.

A thermal initiator, Trigonox™ 21S (0.21 g) was then added to the reactor contents, and the contents were stirred continuously whist the temperature was maintained at 85° C. for 4 hours. The reaction was performed using a nitrogen gas atmosphere throughout.

A second charge of thermal initiator, Trigonox™ 21S (0.21 g), was then added and the polymerisation was continued at a temperature of 85° C. for a further 4 hours, still using a nitrogen gas atmosphere. These steps polymerised the ethylenically unsaturated monomers to prepare the Comparative Polymeric Dispersant solution (CD1).

The reactor contents were then cooled to a temperature of 25° C., poured into a rotary evaporator flask and evaporated to concentrate the Comparative Polymeric Dispersant (CD1) to about 40% by weight. The molecular weights of the Comparative Polymeric Dispersant (CD1) as measured by gel permeation chromatography using a DMF solvent and polystyrene standards were Mn 36,400 and Mw 60,900. Thus, step 3.6 resulted in the preparation of Comparative Polymeric Dispersant solution (CD1).

### 3.7 Polymer Neutralisation

The Comparative Polymeric Dispersant solution (CD1) prepared in 3.6 above (59.1 g) was neutralised by the addition of a solution containing potassium hydroxide (2.61 g) and water (107.7 g) to give a solids content of 15% by weight. This prepared Neutralised comparative dispersant solution (NCD1).

### 4. Preparation of Pigment Dispersions

Pigment dispersions PD1, PD2 and comparative pigment dispersion CPD1 were prepared from neutralised dispersants ND1, ND2 and NCD1 respectively using the following general method:

In each case cyan pigment (TRB 2 from Dainichiseika color and chemicals mfg co ltd), (15 parts), was added to the neutralised dispersant solution (e.g. ND1) (50 parts) and de-ionized water (35 parts). The resulting composition was then mixed using a Red Devil 5400 paint mixer to form a pre-dispersion. The pre-dispersion was then placed into a Branson Digital S450D Ultrasonifier fitted with a 1.25 cm tapped horn with a flat tip. The sample was cooled using an ice-bath and the pigment was dispersed at 60% amplitude to give a resulting pigment dispersion with an MV average particle size of less than 150 nm as measured by a Nanotrac™ particle sizing instrument.

We found that for example, magenta, yellow and black pigment dispersions could also be made by the same method.

### 5. Cross-Linking of Pigment Dispersions

In some cases the above mentioned pigment dispersions were cross-linked to give a dispersion which were more robust, in particular with an improved solvent compatibility. As an example, cyan pigment dispersion (PD2) (97.92 parts) was mixed with Denacol™ EX321 (0.25 parts an epoxy cross-linking agent) and 6.18 weight % boric acid aqueous solution (1.83 parts) and was stirred at 60° C. for 24 hours to complete the cross-linking step. This resulted in cross-linked (XL) pigment dispersion (XLPD2). The resulting dispersion may be used directly to prepare ink jet printing inks or it may optionally, be purified by for example, cross flow membrane filtration.

### 6. Preparation of Inks 1, 2.2A and Comparative Ink 1

Inks 1, 2, 2A and Comparative Ink 1 were prepared by rolling a sealed bottle on rollers for a period of 30 minutes containing the following ingredients: cyan pigment dispersion as indicated in Table 1 (72.5 parts) and an organic solvent mixture (27.5 parts) comprising 2-pyrrolidone (3 parts), glycerol (15 parts), 1,2-hexane diol (4 parts), ethylene glycol (5 parts) and Surfynol® 465 (0.5 parts obtained from Air Products).

### 7. Printing of Inks and Measurement of Optical Density

Inks 1, 2, 2A and Comparative Ink 1 were applied to HP Colorlok™ ink jet paper using an IJ printer and allowed to dry before the reflectance optical density (ROD) was measured using a Gretag Macbeth Spectrolino spectrophotometer.

TABLE 1

| | Dispersion | |
|---|---|---|
| Ink | used | ROD |
| Comparative | CPD1 | 1.24 |
| Ink 1 | | |
| Ink 1 | PD1 | 1.29 |
| Ink 2 | PD2 | 1.29 |
| Ink 2A | XLPD2 | 1.25 |

The results clearly showed that higher optical densities were obtained from dispersants containing the compounds of Formula (1) according to the first aspect of the present invention. Similar results were found on other papers or using other pigments.

1. A compound of Formula (1) or a salt thereof: 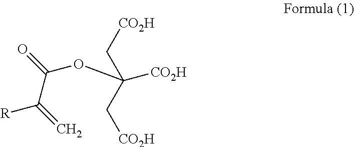
wherein R is H, methyl or ethyl.

2. A compound of Formula (1) according to claim 1 which is in the form of a salt selected from the group consisting of alkali metals, ammonium and substituted ammonium salts or a mixtures thereof.

3. A polymer obtained by polymerising at least a compound of Formula (1) or a salt thereof according to claim 1.

4. A polymer according to claim 3 which is obtained by copolymerising a monomer composition comprising:
i) one or more compounds of Formula (1) or salts thereof;
ii) one or more hydrophobic ethylenically unsaturated monomers; and optionally
iii) one or more hydrophilic ethylenically unsaturated monomers other than component i).

5. A polymer according to claim 4 wherein at least one of the hydrophobic ethylenically unsaturated monomers is selected from the groups consisting of 2-ethyl hexyl (meth)acrylate, lauryl (meth)acrylate, benzyl (meth)acrylate, butyl (meth)acrylate, styrene and methyl (meth)acrylate.

6. A polymer according to claim 4 which is obtained by copolymerising a monomer composition comprising:
i) 1 to 50 parts of one or more compounds of Formula (1) or salts thereof;
ii) 50 to 99 parts of one or more hydrophobic ethylenically unsaturated monomers; and
iii) 0 to 25 parts of one or more hydrophilic ethylenically unsaturated monomers other than component i);
wherein all parts are by weight and the sum of the parts i)+ii)+iii) equals 100.

7. A polymer according to claim 4 wherein at least one of the hydrophilic ethylenically unsaturated monomers is selected from the groups consisting of acrylic acid, methacrylic acid, beta carboxy ethyl acrylate, itaconic acid, crotonic acid, maleic acid or fumaric acid.

8. A polymer according to claim 3 which has no polyethyleneoxy or hydroxy groups.

9. A polymer according to claim 3 having a weight averaged molecular weight of from 5,000 to 200,000.

10. A polymer according to Claim 3 having an acid value of from 50 to 150 mg KOH/g.

11. A polymer according to claim 3 having no hydrophilic groups other than carboxylic acid groups or salts thereof.

12. A dispersion comprising a polymer according to claim 3, a pigment and a liquid medium.

13. A dispersion according to claim 12 wherein the liquid medium is or comprises water.

14. A dispersion according to claim 13 wherein the liquid medium comprises water and a water-miscible organic solvent.

15. An ink comprising a dispersion according to claim 12 and one or more additives selected from the group consisting of viscosity modifiers, pH buffers, metal chelating agents, surfactants, corrosion inhibitors, biocides, dyes, water miscible organic solvent(s) and/or kogation reducing additives.

16. A dispersion according to claim 12 or an ink according to claim 15 wherein the polymer is cross-linked in the presence of the pigment and the liquid medium so as to encapsulate the pigment.

17. A process for preparing a dispersion of a pigment in a liquid medium comprising the steps:
i) providing a mixture comprising the pigment, the liquid medium and a polymer according to claim 3;
ii) mechanically treating the mixture in i) so as to disperse the pigment in the liquid medium.

18. A process according to claim 17 wherein the volume averaged particle size of the pigment particles after the mechanical treatment is from 30 nm to 300 nm.

19. A process according to claim 17 wherein the mechanical treatment is or comprises bead milling, bead shaking, ultrasonication, microfluidization, high pressure homogenisation or a combination thereof.

20. A process for preparing a compound of Formula (1) or a salt thereof as defined in claim 1 which comprises the steps i) to iii) in the following order:
i) protecting all the carboxylic acid groups or salts thereof in citric acid by means of reacting citric acid or a salt thereof with a protecting agent so as to provide a compound of Formula (2a); 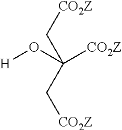
Formula (2a) wherein Z is a protecting group;
ii) esterifying the hydroxy group in the compound of Formula (2a) so as to provide a compound of Formula (2c); 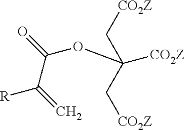
Formula (2c) wherein Z is as hereinbefore defined and R is H, methyl or ethyl;
iii) deprotecting the CO₂Z ester groups in the compound of Formula (2c) as prepared in step ii).
